(19) United States
(12) Patent Application Publication
(10) Pub. No.: US 2009/0326326 A1
(43) Pub. Date: Dec. 31, 2009

(54) Endoscope Head Apparatus

(75) Inventors: Wei Teng Lin, Tainan City (TW); Chih Chun Chan, Miaoli County (TW)

Correspondence Address: WPAT, PC;INTELLECTUAL PROPERTY ATTORNEYS, 2030 MAIN STREET, SUITE 1300, IRVINE, CA, 92614, US

(73) Assignee: MEDICAL INTUBATION TECHNOLOGY CORPORATION, Taoyuan County (TW)

(21) Appl. No.: 12/266,917

(22) Filed: Nov. 7, 2008

(30) Foreign Application Priority Data
Jun. 25, 2008 (TW) 097123792

(51) Int. Cl. A61B 1/005

(52) U.S. Cl. 600/146

(57) ABSTRACT

The present invention discloses an endoscope head apparatus comprising a head mechanism, an elastic element, a support portion, a first wire, a second wire and a bilaterally flexible body. The elastic element is connected to the head mechanism and has a first accommodating space therein. The support portion is connected to the elastic element and has a second accommodating space therein. The first wire and the second wire are connected to head mechanism. The bilaterally flexible body is located at the first accommodating space, and connected to the head mechanism and the support portion respectively, and has a first bendable surface and a second bendable surface. The bilaterally flexible body can swing toward the first surface or the second surface by pulling the first wire or the second wire.

### FIELD OF THE INVENTION

The present invention is related to an endoscope head apparatus, particularly to an endoscope head apparatus which is assembled with an elastic element and a bilaterally flexible body, which can swing bilaterally or unilaterally.

### BACKGROUND OF THE INVENTION

At present, an endoscope head apparatus is extensively applied to examine digestive tracts and organs. Referring to FIG. 1 for a schematic diagram of an endoscope head apparatus 91 of the prior art, front end of an endoscope head apparatus 91 is provided with a head structure 92, a plurality of ring-shaped metals 93 and a plurality of rivets 94. An endoscope lens (not shown in the drawing) is disposed in the head structure 92. Each ring-shaped metal 93 has a first rivet hole 95 and a second rivet hole (not shown in the drawing) facing each other. The ring-shaped metals 93 are connected by penetrating the rivets 94 into the first rivet hole 95 and the second rivet hole. A first wire 96 and a second wire 97 are disposed in these ring-shaped metals 93 and connected to the head structure 92. By pulling the first wire 96 or the second wire 97 respectively, the head structure 92 is pulled to change the direction and further control the endoscope lens, so that the endoscope can be controlled to enter a volume of cavity of human body, such as a stomach, etc. Therefore, most tissue image in the cavity can be observed.

However, because each size of the ring-shaped metals 93, first rivet hole 95, second rivet hole, and rivet 94 of the endoscope head apparatus 91 are required to be precisely achieved under 0.2 mm to maintain a precise structure, it leads that the production becomes tedious, the cost is increased massively, and the price is high.

Besides, because of the restriction of the structure of each ring-shaped metal of the endoscope head apparatus 91 in the prior art, the bend angles are limited, and can't reach big extent, the tissue image in the cavity can't be observed completely, and causes the defect in omission of a certain tissue image. Therefore, the application of conventional bilaterally head of endoscope is greatly limited.

Besides, when the first wire 96 or the second wire 97 which has been pulled is loosened, then bent ring-shaped metal 93 still can't return to the original status and keep at the angle when the wire bodies are pulled. Therefore, the user has to pull the first wire 96 or the second wire 97 to return the ring-shaped metal 93 to the original status. The behavior makes user very inconvenient.

### SUMMARY OF THE INVENTION

Therefore, one of objects of the present invention is to provide an endoscope head apparatus which uses a bendable elastic element and a flexible body to achieve the function of swinging endoscope bilaterally and unilaterally in simple structure.

The other object of the present invention is to provide an endoscope head apparatus which uses a bendable elastic element and a flexible body to avoid the bent angle being interfered by the structure.

Therefore, in order to achieve the said objects, the present invention discloses an endoscope head apparatus comprising a head mechanism, an elastic element, a support portion, a first wire, a second wire, and a bilaterally flexible body. The head mechanism has an endoscope lens, a first connecting portion and a second connecting portion facing each other. The elastic element is connected to the head mechanism. The elastic element has a first accommodating space inside. The support portion is connected to the elastic element and has a second accommodating space inside. The first wire and the second wire are respectively connected to the first connecting portion and the second connecting portion, and pass through the second accommodating space via the first accommodating space. The bilaterally flexible body is located at the first accommodating space and is between the first wire and the second wire.

One end of the bilaterally flexible body is connected to the head mechanism, and the other end is connected to the support portion. The bilaterally flexible body has a first bendable surface and a second bendable surface which are facing the first wire and the second wire respectively. The bilaterally flexible body can swing toward the corresponding first surface and the second surface by pulling the first wire and the second wire. Accepting the said content, the endoscope head apparatus according to the present invention has the following advantages:

(1) The endoscope head apparatus according to the present invention can be manufactured in low cost because of its simple structure comprising the elastic element, bilaterally flexible body and unilaterally flexible body.

(2) The endoscope head apparatus according to the present invention can return to the original place after being bent due to flexibility of the elastic element.

(3) The endoscope head apparatus according to the present invention has a smaller bend radius of curvature by using the bendable elastic element, of the bilaterally flexible body and of the unilaterally flexible body.

(4) The curved direction and position of the endoscope head apparatus according to the present invention can be accurately controlled.

With these and other objects, advantages, and features of the invention that may become hereinafter apparent, the nature of the invention may be more clearly understood by reference to the detailed description of the invention, the embodiments and to the several drawings herein.

### BRIEF DESCRIPTION OF THE DRAWINGS

The exemplary embodiment(s) of the present invention will be understood more fully from the detailed description given below and from the accompanying drawings of various embodiments of the invention, which, however, should not be taken to limit the invention to the specific embodiments, but are for explanation and understanding only.

FIG. 1 is a schematic diagram of an endoscope head apparatus of the prior art.

FIG. 2 is a first schematic diagram of three dimensional structure of a first embodiment of the endoscope head apparatus of the present invention.

FIG. 3 is a second schematic diagram of three dimensional structure of first embodiment of the endoscope head apparatus of the present invention.

FIG. 4 is an activating schematic diagram of the endoscope head apparatus of the present invention.

FIG. 5 is a three dimensional structural diagram of a second embodiment of the endoscope head apparatus of the present invention.

FIG. 6 is a schematic diagram of an appearance of a flexible body of a second embodiment of the endoscope head apparatus of the present invention.

FIG. 7 is a first schematic diagram of three dimensional structure of a third embodiment of an endoscope head apparatus of the present invention.

FIG. 8 is a second schematic diagram of three dimensional structure of a third embodiment of the endoscope head apparatus of the present invention.

FIG. 9 is an activating schematic diagram of a third embodiment of the endoscope head apparatus of the present invention.

### DETAILED DESCRIPTION

Exemplary embodiments of the present invention are described herein in the context of an endoscope head apparatus.

Those of ordinary skilled in the art will realize that the following detailed description of the exemplary embodiment(s) is illustrative only and is not intended to be in any way limiting. Other embodiments will readily suggest themselves to such skilled persons having the benefit of this disclosure. Reference will now be made in detail to implementations of the exemplary embodiment(s) as illustrated in the accompanying drawings. The same reference indicators will be used throughout the drawings and the following detailed description to refer to the same or like parts.

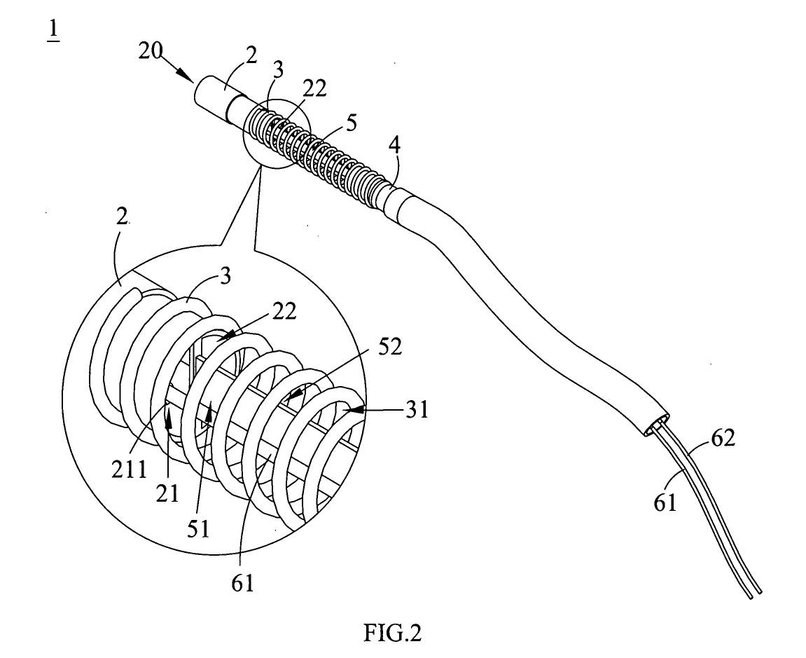

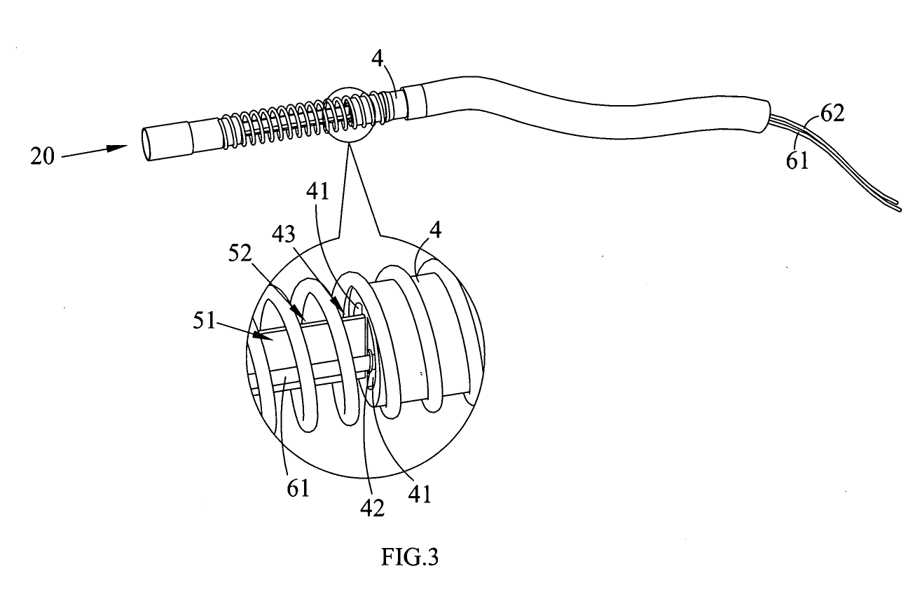

Referring to FIG. 2 and FIG. 3 for a first schematic diagram and a second schematic diagram of three dimensional structure of first embodiment of an endoscope head apparatus of the present invention, an endoscope head apparatus 1 comprises a head mechanism 2, an elastic element 3, a support portion 4, at least a bilaterally flexible body 5, a first wire 61, and a second wire 62. The head mechanism 2 has an endoscope lens 20, a first connecting portion 21 and a second connecting portion 22 facing each other. Preferably, the first connecting portion 21 and the second connecting portion 22 can be a first connecting hole 211 and a second connecting hole (not shown in the drawing), or the first connecting portion 21 and the second connecting portion 22 can further wedge the first wire 61 and the second wire 62 onto the corresponding first connecting hole 211 and the second connecting hole by welding manner. Preferably, the elastic element 3 is a spring, which is connected on the head mechanism 2. The elastic element 3 has a first accommodating space 31 inside. The support portion 4 is connected on the elastic element 3 and has a first through hole 42, a second through hole 43 facing each other and a second accommodating space 41 inside. The first through hole 42 and the first connecting portion 21 are located at the same side. The second through hole 43 and the second connecting portion 22 are located at the same side. Preferably, the head mechanism 2 and the support portion 4 are ring-shaped metals.

Preferably, the first wire 61 and the second wire 62 can be either metal wires or plastic wires, which can be connected to and fixed at the first connecting portion 21 and the second connecting portion 22. The first wire 61 and the second wire 62 respectively pass through the first through hole 42 and the second through hole 43 to control the bend direction of the first surface 51 and the second surface 52. The first wire 61 and the second wire 62 pass through the second accommodating space 41 via the first accommodating space 31.

Preferably, the bilaterally flexible body 5 can be made of a material of bilaterally bent metal or plastic. Preferably, the shape of the bilaterally flexible body 5 can be a bilaterally bent flat form, or a flat bamboo node form, or a rectangle form, or an irregular form, or a cylinder form, or a cylindrical bamboo node form. In this embodiment, the bilaterally flexible body 5 is performed by the bilaterally bent flat form.

The bilaterally flexible body 5 is located inside the first accommodating space 31. Depending on demand, the bilaterally flexible body 5 can be disposed at one side or the center or two sides of the first accommodating space 31. One end of the bilaterally flexible body 5 is fixed at the head mechanism 2, and the other end is fixed at the support portion 4, so as to prevent the bilaterally flexible body 5 from taking itself as a rotation axis to rotate. The bilaterally flexible body 5 has a first bendable surface 51 and a second bendable surface 52. The first surface 51 and the first wire 61 are located at the same side, and the second surface 52 and the second wire 62 are located at the same side. The bilaterally flexible body 5 can respectively swing toward the first surface 51 and the second surface 52 by pulling the first wire 61 and the second wire 62.

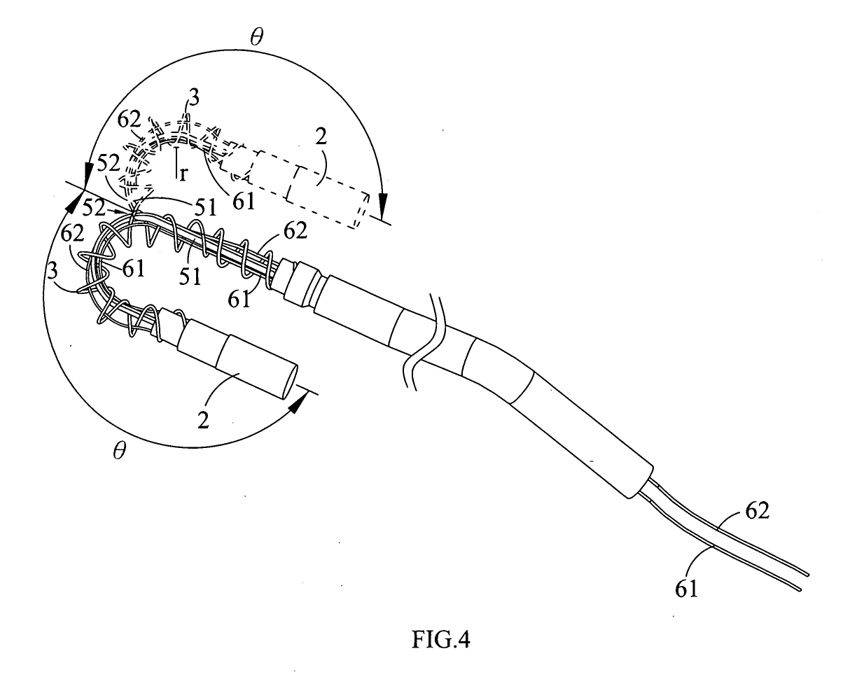

FIG. 4 illustrates an schematic view of activating status of the endoscope head apparatus of the present invention, when the first wire 61 connected to the head mechanism 2 is pulled, the two ends of the bilaterally flexible body 5 are fixed at the head mechanism 2 and the support portion 4, the bilaterally flexible body 5 is prevented from taking itself as an axis to rotate, so the first surface 51 is focused to bend as an angle θ and the elastic element 3 is also bent as an angle θ according to the first surface 51. The angle θ is ranged between 0° and 180°. Similarly, when the second wire 62 connected to the head mechanism 2 is pulled, the second surface 52 is focused to bend an as angle θ and the elastic element 3 is also bent as an angle θ according to the first surface 52. The angle θ is ranged between 0° and 180°. Because the first surface 51, the second surface 52 and the elastic element 3 are bent with smaller radius r of curvature, the tissue image in the cavity can be completely observed, and the defect of omitting a certain tissue image in the cavity can be prevented. Therefore, the application of the endoscope head apparatus 1 is not limited by its structure. Once the pulled first wire 61 or second wire 62 is loosened, the head mechanism 2 then follows the bent elastic element 3 and the bilaterally flexible body 5 to return to the original status, and does not keep at the angle and the position while being pulled. Therefore, the apparatus won't be very inconvenient to the user.

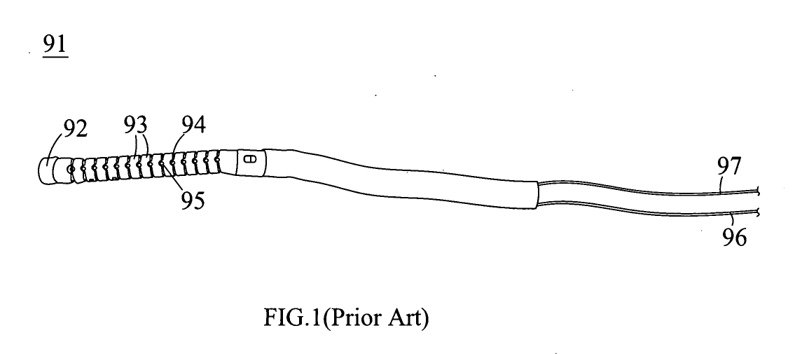

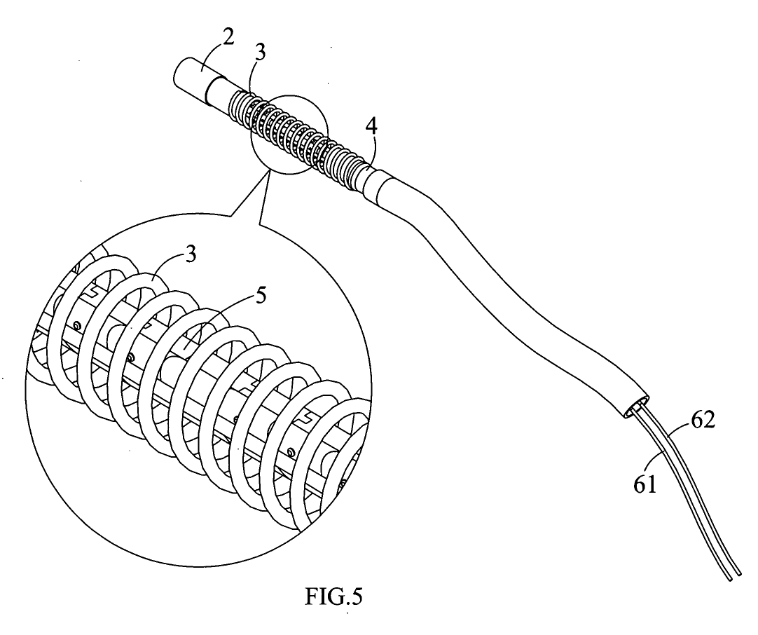

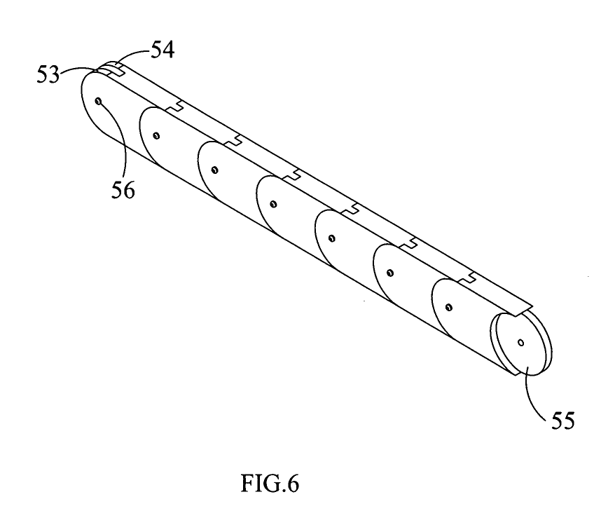

FIG. 5 and FIG. 6 illustrate a three dimensional diagram of the structure of the second embodiment of the endoscope head apparatus of the present invention, and a schematic diagram of the flexible body of the second embodiment of the endoscope head apparatus of the present invention, the structures of the head mechanism 2, the elastic element 3, the support portion 4, the first wire 61 and the second wire 62 of the second embodiment are approximately similar to that of first embodiment. The difference between the second embodiment and first embodiment is that the bilaterally flexible body 5 in the flexible element 3 comprises a plurality of flat bamboo nodes or cylindrical bamboo nodes. In second embodiment, the bamboo node type flat form is taken as example for description. One end of bamboo node type flat form has a first protrusion 53 and a second protrusion 54, and the other end has a third protrusion 55. The third protrusion 55 of a bamboo node type flat form is disposed between the first protrusion 53 and the second protrusion 54 of another bamboo node type flat form. The first protrusion 53, the second protrusion 54, the third protrusion 55 are connected by a rotary axis 56, so that each connected bamboo node type flat form can swing bilaterally by depending the rotary axis 56 as an rotation axis. The rest structure and effect of second embodiment shown in FIG. 5 are similar as FIG. 1, FIG. 2 and FIG. 3, therefore, the specification of the identical portion is omitted, and we no longer go into detailed description.

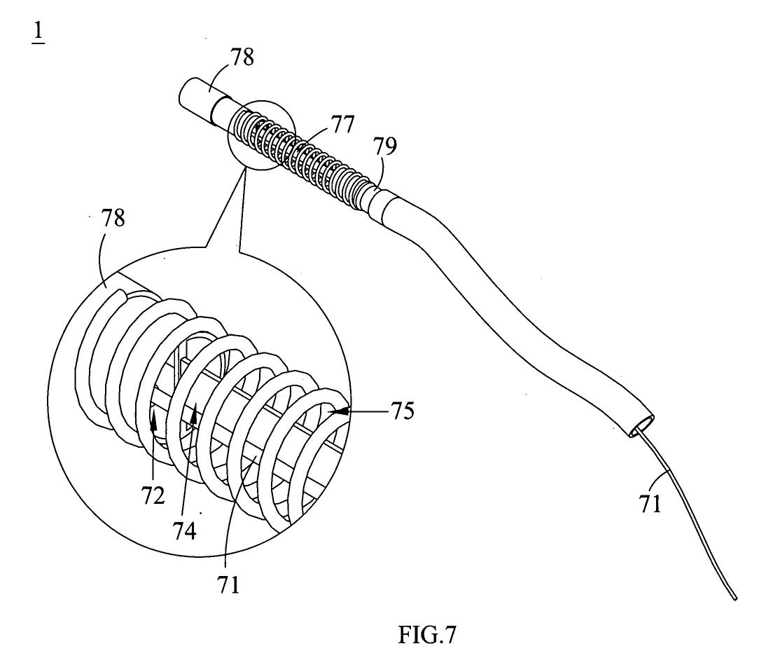

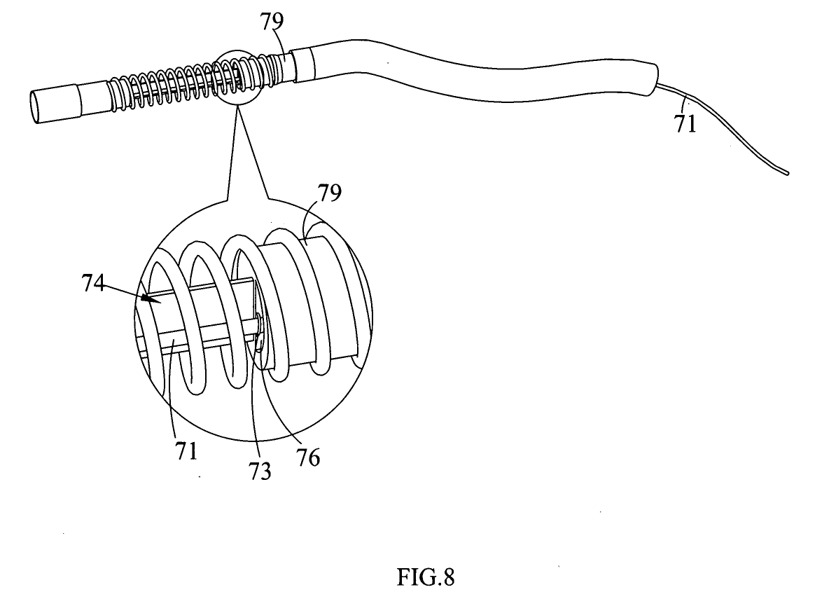

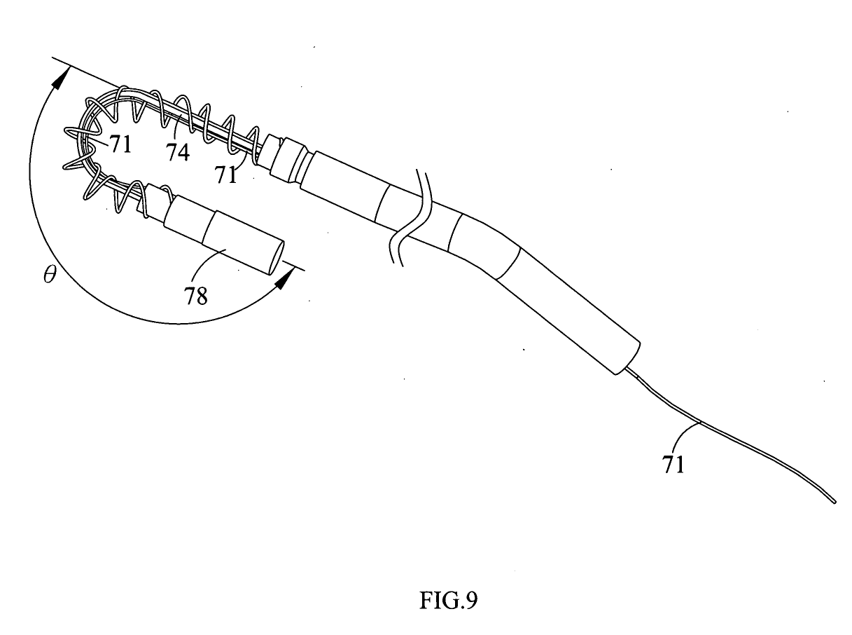

FIG. 7, FIG. 8 and FIG. 9 illustrates a first schematic diagram, a second schematic diagram, and an activating schematic diagram of three dimensional structure of the third embodiment of the endoscope head apparatus of the present invention. The third embodiment differs from the first embodiment and the second embodiment in that the endoscope head apparatus of third embodiment comprises at least one unilaterally flexible body 77 and a first wire 71. The first wire 71 is respectively connected to a connecting portion 72, and pass through the first through hole 73 to control the bend direction of the first surface 74. The first wire 71 passes through a second accommodating space 76 via a first accommodating space 75. Preferably, the first wire bodies 71 can be a metal wire or a plastic wire.

Preferably, the unilaterally flexible body 77 can be a unilaterally bent metal wire or a plastic wire. Preferably, the shape of the unilaterally flexible body 77 can be a unilaterally bent flat form, or a bamboo node type flat form, or a rectangle form, or an irregular form, or a cylinder form, or a bamboo node type cylinder form. In the embodiment, the unilaterally flexible body 77 is illustrated by the unilaterally bent flat form.

The unilaterally flexible body 77 is located in the first accommodating space 75. Preferably, the unilaterally flexible body 77 can be disposed at one side or the center of the first accommodating space 75. One end of the unilaterally flexible body 77 is fixed at the head mechanism 78, and the other end is fixed at the support portion 79, so as to prevent the unilaterally flexible body 77 from taking itself as an axis to rotate. The unilaterally flexible body 77 has a first bendable surface 74. The first surface 74 and the first wire 71 are located at the same side. The unilaterally flexible body 77 can swing unilaterally toward the first surface 74 corresponding to the unilaterally flexible body 77 by pulling the first wire 71.

While particular embodiments of the present invention have been shown and described, it will be obvious to those skilled in the art that, based upon the teachings herein, changes and modifications may be made without departing from this invention and its broader aspects. Therefore, the appended claims are intended to encompass within their scope of all such changes and modifications as are within the true spirit and scope of the exemplary embodiment(s) of the present invention.

What is claimed is:

1. An endoscope head apparatus, comprising:
a head mechanism having an endoscope lens, a first connecting portion and a second connecting portion facing each other;
an elastic element connected to the head mechanism, and having a first accommodating space therein;
a support portion connected to the elastic element, and having a second accommodating space therein;
a first wire and a second wire respectively connected to the first connecting portion and the second connecting portion, and the first wire and the second wire passing through the second accommodating space via the first accommodating space; and
at least one bilaterally flexible body located at the first accommodating space, and having a first bendable surface and a second bendable surface facing each other, and one end of the bilaterally flexible body fixed to the head mechanism, and the other end fixed to the support portion;
wherein, the bilaterally flexible body respectively swings toward the corresponding first surface and the second surface by pulling the first wire and the second wire respectively.

2. The endoscope head apparatus of claim 1, wherein the first connecting portion is fixed at one end of the first wire, and the second connecting portion is fixed at one end of the second wire.

3. The endoscope head apparatus of claim 1, wherein the support portion has a first through hole and a second through hole facing each other, and the first through hole and the first connecting portion are located at the same side, the second through hole and the second connecting portion are located at the same side.

4. The endoscope head apparatus of claim 3, wherein the first wire and the second wire respectively pass through the first through hole and the second through hole for controlling the bend direction of the first surface and the second surface.

5. The endoscope head apparatus of claim 1, wherein the material of the bilaterally flexible body is bilaterally bent metal or plastics.

6. The endoscope head apparatus of claim 1, wherein the elastic element is one of flexible materials comprising a spring, a flexible hose and a braided hose, and the elastic element wraps the bilaterally flexible body and maintains tube structure of the bilaterally flexible body.

7. The endoscope head apparatus of claim 1, wherein the head mechanism and the support portion are ring-shaped metals.

8. The endoscope head apparatus of claim 1, wherein the first wire and the second wire are metal wires or plastic wires.

9. The endoscope head apparatus of claim 1, wherein the bilaterally flexible body is a bilaterally bent flat form, or a flat bamboo node form, or a rectangle form, or an irregular form, or a cylinder form, or a cylindrical bamboo node form.

10. The endoscope head apparatus of claim 9, wherein when the bilaterally flexible body is the flat bamboo node form or the cylindrical bamboo node form, the bilaterally flexible body comprises a plurality of bamboo nodes, and one end of the bamboo node has a first protrusion and a second protrusion, and the other end of the bamboo node has a third protrusion.

11. An endoscope head apparatus, comprising:
a head mechanism having an endoscope lens, and a first connecting portion;
an elastic element connected to the head mechanism and having a first accommodating space therein;
a support portion connected to the elastic element and having a second accommodating space therein;
a first wire connected to the first connecting portion and passing through the second accommodating space via the first accommodating space; and
at least a unilaterally flexible body located in the first accommodating space, and one end of the unilaterally flexible body fixed to the head mechanism, and the other end fixed to the support portion;
wherein the unilaterally flexible body swings correspondingly by pulling the first wire.

12. The endoscope head apparatus of claim 11, wherein the first connecting portion is fixed at one end of the first wire.

13. The endoscope head apparatus of claim 11, wherein the support portion has a first through hole located at the same side as the first connecting portion.

14. The endoscope head apparatus of claim 13, wherein the first wire passes through the first through hole for controlling the bend direction of the first surface.

15. The endoscope head apparatus of claim 11, wherein the material of the unilaterally flexible body is unilaterally bent metal or plastics.

16. The endoscope head apparatus of claim 11, wherein the elastic element is one of flexible materials comprises a spring, a flexible hose and a braided hose, and the elastic element wraps the unilaterally flexible body and maintains the tube structure of the unilaterally flexible body.

17. The endoscope head apparatus of claim 11, wherein the head mechanism and the support portion are ring-shaped metals.

18. The endoscope head apparatus of claim 11, wherein the first wire is a metal wire or a plastic wire.

19. The endoscope head apparatus of claim 11, wherein the unilaterally flexible body is a unilaterally bent flat form, or a flat bamboo node form, or a rectangle form, or an irregular form, or a cylinder form, or a cylindrical bamboo node form.

20. The endoscope head apparatus of claim 19, wherein when the bilaterally flexible body is the flat bamboo node form or the cylindrical bamboo node form, the bilaterally flexible body comprises a plurality of bamboo nodes, and one end of the bamboo node has a first protrusion and a second protrusion, and the other end of the bamboo node has a third protrusion.
